(19) United States
(12) Patent Application Publication
(10) Pub. No.: US 2024/0115647 A1
(43) Pub. Date: Apr. 11, 2024

(54) COMPOSITION COMPRISING FLAVANOL MONOMERS AND E-VINIFERIN

(71) Applicant: ACTIV'INSIDE, BEYCHAC ET CAILLAU (FR)

(72) Inventors: David GAUDOUT, CARIGNAN DE BORDEAUX (FR); Stéphane REY, MONTELIMAR (FR); Benoit LEMAIRE, LIBOURNE (FR); Line POURTAU, CESTAS (FR); Camille POUCHIEU, GABARNAC (FR); Séverine DUBREUIL, TREILLIERES (FR); Benjamin MORAS, FLOIRAC (FR); Mégane BORNERIE, PESSAC (FR); Julien GABASTON, VILLENAVE D'ORNON (FR); Manon ROLAIT, ARVEYRES (FR); Astrid DE VULPILLIERES, BORDEAUX (FR)

(21) Appl. No.: 18/028,097

(22) PCT Filed: Sep. 27, 2021
(86) PCT No.: PCT/EP2021/076536

§ 371 Date: Nov. 30, 2023

(30) Foreign Application Priority Data
Sep. 29, 2020 (FR) FR2009924

(51) Int. Cl. A61K 36/87; A61K 8/31; A61K 8/34; A61K 8/49; A61K 8/9789; A61K 9/00; A61K 31/015; A61K 31/05; A61K 31/343; A61K 31/353; A61K 36/484; A61P 43/00; A61Q 19/10

(52) U.S. Cl. CPC A61K 36/87; A61K 8/31; A61K 8/347; A61K 8/4973; A61K 8/498; A61K 8/9789; A61K 9/0056; A61K 9/0095; A61K 31/015; A61K 31/05; A61K 31/343; A61K 31/353; A61K 36/484; A61P 43/00; A61Q 19/10

(57) ABSTRACT

The present invention relates to a composition comprising the association of the molecules of flavanol monomers and ε-viniferin as a nutritional product or a drug for humans or animals.

### TECHNICAL FIELD

The invention relates to a composition comprising the association of flavanol monomers and ε-viniferin as a nutritional product or a drug for humans or animals.

### PRIOR ART

The endothelium is a single-cell layer that lines the interior of blood vessels and acts as a physical barrier between the tissue layer and the circulating phase. In particular, it makes it possible to regulate vascular tone. Dysfunction of endothelial function, or endothelial dysfunction, is characterized by a reduction in the capacity of the vessels to expand in response to an increase in blood flow. It results mainly from a reduction in the availability of nitrogen monoxide (NO), which may be due either to a reduction in NO production, or to increased capture of same, in particular because of the phenomenon of oxidative stress.

Endothelial function is evaluated by measuring flow-mediated vasodilation (often referred to as “FMD” for flow-mediated dilation).

The endothelium therefore plays a pivotal role in maintaining blood flow (both at the macrovascular and microvascular level). Consequently, the deterioration of endothelial function predominantly leads to cardiovascular diseases or disorders, in particular atherosclerosis, but also vascular diseases, eye diseases, diseases associated with cognitive decline, intestinal functional disorders, erectile dysfunction, or disorders associated with the menopause.

Endothelial dysfunction is known to be an early predictive marker for cardiovascular diseases, which precedes the clinical manifestations thereof. However, cardiovascular diseases constitute the number one cause of death in the world (30% of deaths globally) and represent a major public health problem.

There is therefore a need to identify a product capable of improving endothelial function and making it possible in particular to contribute to the prevention of cardiovascular diseases.

Furthermore, endothelial dysfunction has also been described as a predictive marker of cognitive decline, including in the absence of cerebrovascular disease (Naiberg et al. Psychosom Med. 2016; 78(2):192-207). Indeed, the cerebral blood flow, which plays an essential role in cognitive functions, is closely linked to endothelial function (Fouda et al. Arterioscler Thromb Vasc Biol. 2019 April; 39(4):593-602).

Preserving good vascular health (and therefore optimal endothelial function) is also important for sight. Indeed, cardiovascular diseases (diabetes, hypertension but also hypercholesterolemia) can cause different eye diseases such as hypertensive retinopathy, diabetic retinopathy, glaucoma, etc. (Singh et al. Eur J Ophthalmol. 2020 Apr. 27:1120672120914232).

Endothelial dysfunction is also considered to be one of the etiological factors of inflammatory bowel diseases (Cibor et al. World J Gastroenterol. 2016 Jan. 21; 22(3): 1067-1077).

Furthermore, NO, which is a vasodilator, also plays an essential role in triggering erections. Thus, in men, endothelial dysfunction is often associated with erectile dysfunction (Kovács I et al. J.Cardiovasc Pharmacol, 2008 Feb.; 51(2):148-53).

In women, estrogen stimulates NO production by endothelial cells. However, during the menopause, the drop in estrogen leads to a significant reduction in FMD, reflecting endothelial dysfunction (Somani YB et al. Am J Physiol Heart Circ Physiol, 2019 Aug. 1; 317(2):H395-H404). Furthermore, it has been demonstrated that mental stress induces deterioration of endothelial function (Takase B et al. Clin Cardiol. 2004 Apr.; 27(4):223-7). Likewise, a sleep deficit and certain sleep disorders may adversely affect endothelial function (Budhiraja et al. J Clin Sleep Med. 2007 Jun. 15; 3(4):409-15). Endothelial function, just like microcirculation, is also affected by UV exposure (Wolf et al. Exp Physiol. 2019; 104(7): 1136-1146).

Thus, endothelial dysfunction is the cause of, or is involved in, numerous pathologies.

There is therefore a need to maintain or improve endothelial function.

Several clinical studies have demonstrated a significant improvement in FMD after the consumption of flavonoid-rich food or beverages. Among the flavonoids, it has been demonstrated that the consumption of a single dose of pure epicatechin made it possible to induce a significant increase in FMD within 2 hours (Shafabakhsh et al., Crit Rev Food Sci Nutr. 2020; 60(14):2369-2378).

Furthermore, epidemiological studies have revealed an inverse correlation between the dietary intake of flavonoids and age-related cognitive decline. In particular, it has been demonstrated that flavanol monomers (catechins and epicatechins) exert favorable effects on cognitive functions, and that these effects are essentially due to the capacity of the flavanol monomers to increase cerebral blood flow (Haskell-Ramsay et al., Nutrients. 2018 Jul. 27; 10(8):986). Studies evaluating the effects of epicatechin on cognitive functions have demonstrated favorable effects when the epicatechin dose was more than 50 mg/day, for at least 28 days, in subjects over 50 years old (Haskell-Ramsay et al., Nutrients. 2018 Jul. 27; 10(8):986).

A significant increase in FMD (and therefore an improvement in endothelial function), an improvement in cerebral blood flow and in certain areas of cognitive performance has also been demonstrated after supplementation with resveratrol (Cicero et al. Arch Med Sci. 2019; 15(4):936-943). Pre-clinical experiments suggest that resveratrol dimers, in particular ε-viniferin and δ-viniferin, are capable of inducing the same effects (Wu et al. The Kaohsiung Journal of Medical Sciences 36 (2020): 535-542).

Thus, the beneficial effects of flavonoids, resveratrol or its dimers, individually, on endothelial function have already been reported.

However, there is still a significant need for a product capable of preventing or treating endothelial dysfunction and associated diseases, sleep disorders, stress, cognition or solar protection with improved efficacy compared to the prior art.

### SUMMARY OF THE INVENTION

Thus, the aim of the invention is to provide a novel composition having greater efficacy than existing products in combating diseases associated with endothelial dysfunction compared to said specific molecules consumed individually.

The inventors have surprisingly identified a composition comprising a specific combination of molecules of natural origin as well as a specific amount of said molecules present in said composition, having a synergistic effect and therefore improved efficacy, on endothelial function. They have also demonstrated the benefit of said composition for its antioxidant effects, as well as for protecting the skin from UVB (ultraviolet B) radiation, preventing sunburn, preventing hyperpigmentation, preventing and/or reducing age-related brown spots. Finally, the composition is also of interest for improving memory and/or attention and/or concentration and/or alertness and/or vigilance and/or learning and/or language and/or mood and/or stress and/or anxiety and/or sleep.

In particular, a subject of the invention is a composition specifically comprising at least one molecule belonging to the flavonoid family and at least one molecule belonging to the stilbene family. Such a composition is intended to be used as nutritional product, dietary supplement or drug in humans or animals.

The invention specifically relates to a composition comprising a mixture of molecules, comprising:
at least 15% of flavanol monomers, the percentage being given by dry weight relative to the total dry weight of the composition, and
at least 15 ppm of epsilon-viniferin (ε-viniferin) by dry weight relative to the total dry weight of the composition.

Such a composition thus has a synergistic effect compared to said molecules administered in isolation.

According to a preferred subject of the invention, the amount of flavanol monomers is greater than the amount of ε-viniferin. Preferentially, the ratio of flavanol monomers to ε-viniferin is between 20,000 and 4.

The flavanol monomers and the ε-viniferin can be derived from a plant extract; preferentially, they are derived from at least one Vitis vinifera (grape) extract and/or a mixture of at least two Vitis vinifera extracts.

The composition may be administered orally a as nutritional product, dietary supplement or drug. The composition and/or one or more molecules of the composition may be encapsulated or microencapsulated in a food support. The composition may be in the form of a powder, gelcap, tablet, capsule, solution, suspension, emulsion or chewing gum.

When the composition is intended to be used as a drug or as a dietary supplement, the composition is preferentially used for preventing and/or treating diseases selected from cardiovascular diseases, vascular diseases, diseases associated with cognitive decline, diseases associated with memory loss, neurodegenerative diseases, digestive diseases, joint diseases, erectile diseases and disorders associated with the menopause.

Another aspect of the invention relates to a non-therapeutic use of the composition according to the invention in healthy humans or animals, for improving cognitive functions and/or executive functions, and/or for limiting normal age-related cognitive decline and/or for improving memory and/or attention and/or concentration and/or alertness and/or vigilance and/or learning and/or language and/or mood and/or stress and/or anxiety and/or sleep and/or for homogenizing skin pigmentation, and/or for reducing the appearance of age-related skin blemishes and/or for regulating melanogenesis and/or for preventing skin hyperpigmentation and/or for preventing sunburn and/or for improving the brightness of the complexion.

Other features and advantages will emerge from the detailed description of the invention, examples and figures that follows.

### BRIEF DESCRIPTION OF THE FIGURES

FIG. 1 shows, during the evaluation of tyrosinase activity, the measure of absorbance (normalized and corrected) (average±SEM) at 7 min based on the different compositions tested and also on their respective inhibition percentages relative to the control. n: number of duplicates. Concentrations tested: red grape pomace extract: 0.025 mg/ml, licorice extract: 0.0375 mg/ml, grape seed extract: 0.0286 mg/ml, a mixture comprising a composition according to the invention: 0.1 mg/ml. The mixture was composed of 58.8% of a composition according to the invention, itself composed of 53.4% of E1 and 46.6% of E3 (the composition according to the invention providing 19.4% of flavanol monomers and 28 ppm of ε-viniferin) and 41.2% of a licorice extract providing 8.1% of glycyrrhizic acid. p value for red grape pomace extract: <0.724, p value for licorice extract: <0.0001, p value for grape seed extract: <0.0001, p value for the interaction of the 3 extracts: <0.0001.

FIG. 2 shows, in A: the % vasorelaxation observed (average±SEM) or estimated based on the cumulative sample volumes added to the component vessel. The curves shown demonstrate the effect of a mixture tested at 2 mg/ml, the estimated effect of a seed extract (E1) at 1.1 mg/ml, and the estimated effect of a white grape pomace extract (E5) at 0.1 mg/ml. The mixture tested comprises 65% of a composition according to the invention, itself composed of 92.3% of E1 and 7.7% of E5 (the composition according to the invention providing 33% of flavanol monomers and 459 ppm of ε-viniferin) and 35% of maltodextrin. The estimated effect of a 1.1 mg/ml seed extract was compared to observed data beforehand. Statistical analysis using the t-test did not show any significant difference between the estimated values and the observed values for cumulative volumes of between 100 μl and 2000 μl. in B: a histogram showing the % vasorelaxation (average±SEM) for 300 μl of samples added to the component vessel.

FIG. 3 shows the mean fluorescence intensity (MFI) (average±SEM) emitted by the cells labeled with CM-H2DCFDA. Mixture of extracts: mixture tested at 270.9 mg/ml and comprising: 53.7% of a composition according to the invention, itself composed of 53.4% of E1, and 46.6% of E3 (the composition according to the invention providing 19.4% of flavanol monomers and 28 ppm of ε-viniferin) and 37.5% of a licorice extract. ***p<0.001.

FIG. 4 shows the keratinocyte proliferation rate expressed in % (relative to the non-irradiated control for each condition, mean±SEM). Mixture of extracts: mixture tested at 270.9 mg/ml and comprising: 53.7% of a composition according to the invention, itself composed of 53.4% of E1, and 46.6% of E3 (the composition according to the invention providing 19.4% of flavanol monomers and 28 ppm of ε-viniferin) and 37.5% of a licorice extract. ***p <0.001.

### DETAILED DESCRIPTION OF THE INVENTION

### Definition

For the purposes of the invention, “animal” means any animal with the exclusion of humans.

For the purposes of the invention, “Vitis vinifera extract” or “grape extract” means an extract comprising at least one molecule, in particular at least flavanol monomers and/or ε-viniferin, preferentially a set of molecules, obtained from Vitis vinifera and/or a by-product of Vitis vinifera. The raw material may be leaves and/or fruit and/or seeds and/or skins and/or stalks and/or woody parts (vine shoots, vine stocks, roots), and/or pomace and/or wine; preferentially, the raw material is grape pomace (comprising seeds, skins and optionally stalks) and/or grape seed.

“Ppm” means parts per million in the mixture.

For the purposes of the invention, “nutritional product” means a food ingredient for nutritional purposes used alone or combined with other food ingredients or additives in food formulations including dietary supplements and food intended for humans or animals.

For the purposes of the invention, “drug” means an active agent used for therapeutic purposes used alone or combined with other active or non-active substances in medicinal formulas, including for phytotherapy or dietary supplements having a therapeutic effect intended for humans or animals.

For the purposes of the invention, “preventing” or “prevention” means reducing, to a lower level, the risk or probability of a given phenomenon occurring, that is to say in the context of the present invention, of a disease or disorder.

For the purposes of the invention, “treating” or “treatment” means reducing the progression of the disease or disorder, stabilizing, reversing or regressing, or even interrupting or inhibiting the progression of a disease or disorder. In the context of the invention, these terms also apply to one or more symptoms of said diseases or disorders of the present invention.

### Composition

The subject of the present invention is therefore a novel composition having in particular increased efficacy in improving or maintaining endothelial dysfunction and in solar protection.

To this end, the invention targets a composition comprising a mixture of molecules, comprising:
at least 15% of flavanol monomers, the percentage being given by dry weight relative to the total dry weight of the composition, and
at least 15 ppm of ε-viniferin by dry weight relative to the total dry weight of the composition.

Flavanol monomers are compounds of the flavonoid family, in particular of the subclass of the flavanols, comprising in particular flavan-3-ols or flavanols or catechins. The most abundant class consists of catechins and/or epicatechins. These are powerful antioxidants which help in particular to prevent inflammatory and coronary diseases but also to maintain or increase cognitive performance. In the context of the invention, the flavanol monomers are selected from catechins, epicatechins, and also their allylated forms, such as epicatechin-3-O-gallate, epigallocatechin, epicatechin gallate, epigallocatechin gallate. These monomers can be extracted from one or more plants (all or part) such as tea (Camelia sinensis), apple (Malus domestica), cacao (Theobroma cacao) or derived from plants (all or part) belonging to the genus Vitis and preferentially Vitis vinifera. They may also be derived from microalgae.

ε-viniferin is a natural phenol of the stilbene family, in particular of the stilbenoids. ε-viniferin is also a resveratrol dimer. It can be extracted from one or more plants such as plants (all or part) belonging to the genus Iris—Iridaceae; Sophora—Fabaceae; Gnetum—Gnetaceae; Carex—Cyperaceae; peony (Paeonia)—Paeoniaceae; Dipterocarpus—Dipterocarpaceae and more particularly belong to the genus Vitis, preferentially Vitis vinifera. ε-viniferin may also be derived from microalgae.

According to a subject of the invention, the composition preferentially comprises at least 100 ppm of stilbenes comprising said at least 15 ppm of ε-viniferin. Thus, the composition according to the invention comprises at least 100 ppm of stilbenes, including at least 15 ppm of ε-viniferin. In other words, the composition may comprise, for example, 85 ppm of stilbenes other than ε-viniferin and 15 ppm of ε-viniferin. Said other stilbenes may be pinosylvine, piceatannol, trans-resveratrol, trans-pterostilbene, rhapontigenin, isorhapontigenin, rhapontin, ponticin, trans-piceid or astringin. (delta and/or omega and/or alpha and/or R and/or R2) viniferin, ampelosin A and/or E and/or F and/or H, miyabenol C, parthenocisin A, pallidol, vitisin C, hopeaphenol, isohopeaphenol, viniferol E.

According to a preferred embodiment, the composition according to the invention may comprise at least 20% of flavanol monomers, the percentage being given by dry weight relative to the total dry weight of the composition, more preferentially at least 25% of flavanol monomers.

According to another embodiment, the composition according to any one of the preceding embodiments may comprise at least 50 ppm of ε-viniferin by dry weight relative to the total dry weight of the composition, more preferentially at least 100 ppm of ε-viniferin.

According to another preferred subject, the inventors have identified that the composition according to the invention has improved activity when the amount by weight of flavanol monomers is greater than the amount by weight of stilbene, preferentially ε-viniferin.

Preferentially, the ratio of flavanol monomers to ε-viniferin is between 20,000 and 4. The term “ratio” means the magnitude relationship between the amount of flavanols and the amount of ε-viniferin (flavanol monomers/ε-viniferin). In particular, the ratio is between 15,000 and 30, even more preferentially between 10,000 and 100.

The flavanol monomers are preferably of natural origin. These molecules can thus be obtained from a natural product, preferentially from a plant extract, more preferentially selected from a Vitis extract, preferentially Vitis vinifera, a tea (Camelia sinensis) extract, an apple (Malus domestica) extract, a cacao extract (Theobroma cacao). They may also be derived from microalgae.

ε-viniferin is of natural origin and can thus be obtained from a natural product, preferentially from a plant extract, more preferentially selected from a Vitis extract, preferentially Vitis vinifera.

When the flavanol monomers and ε-viniferin are obtained from the same plant, the plant is preferentially a Vitis extract, more preferentially a Vitis vinifera extract.

It is already known that grapes and wine comprise flavanol monomers. However, the presence of stilbenes, and in particular of ε-viniferin, is not systematic, as indicated in Table 1 below. In addition, the concentration of monomers from Vitis vinifera naturally never exceeds 10% of the total polyphenols.

However, the composition according to the invention targets a mixture of molecules comprising at least 15% of flavanol monomers. Thus, the origin and type of raw material that makes it possible to obtain the extract(s) and also the method applied that makes it possible to obtain it/them are combined in order to obtain a mixture of molecules comprising at least 15% of flavanol monomers and at least 15 ppm of ε-viniferin. The concentrations of molecules based on different raw grape materials are given in Table 1 below.

TABLE 1

| | | | | | | Stilbenes | | | |
|---|---|---|---|---|---|---|---|---|---|
| | Total | Flavanol monomers | | | | Of which | | | |
| Vitis | polyphenols | Catechin | Epicatechin | | [C + EC]/ | trans- | | Of which | |
| vinifera | (TP) (Folin) | (C) | (EC) | Total C + EC | PT | resveratrol | | ε-viniferin | |
| Grape juice | | 1.21 | 1.26 | 2.47 | | 0.0508 | ppm | — | |
| (mg/100 ml)/ | | | | (0.002%) | | | | | |
| (%) or ppm | | | | | | | | | |
| Black grapes | 184.97 | 5.46 | 5.24 | 10.7 | 5.8% | 1.5 | ppm | — | |
| (mg/100 g)/ | (0.18%) | | | (0.01%) | | | | | |
| (%) or ppm | | | | | | | | | |
| White grapes | 121.80 | 1.41 | 0.49 | 1.90 | 1.6% | 0.3 | ppm | — | |
| (mg/100 g)/ | (0.12%) | | | (0.002%) | | | | | |
| (%) or ppm | | | | | | | | | |
| Red wine | 215.48 | 6.81 | 3.78 | 10.59 | 4.9% | 1.8 | ppm | 1.5 | ppm |
| (mg/100 m) | (0.22%) | | | (0.01%) | | | | | |
| (%) or ppm | | | | | | | | | |
| Rosé wine | 82.21 | 0.91 | 0.55 | 1.46 | 1.8% | 0.6 | ppm | — | |
| (mg/100 m)/ | (0.08%) | | | (0.001%) | | | | | |
| (%) or ppm | | | | | | | | | |
| White wine | 32.10 | 1.08 | 0.95 | 2.03 | 6.3% | 0.3 | ppm | 0.0557 | ppm |
| (mg/100 ml)/ | (0.03%) | | | (0.002%) | | | | | |
| (%) or ppm | | | | | | | | | |
| Invention | >50% | | | >15% | >30% | | | 15 | ppm |

According to a preferred embodiment, the Vitis vinifera extract(s) in the composition according to the invention, when it contains same, comprise(s) at least 50% total polyphenols, the percentage being given by dry weight relative to the total weight of the Vitis vinifera extract.

Preferentially, the Vitis vinifera extract is a grape pomace extract and/or a grape seed extract; more preferentially, the composition comprises at least one grape seed extract and/or a grape pomace extract. According to one embodiment, the composition according to the invention comprises at least one grape seed extract and a grape pomace extract.

In addition to the flavanol monomers and ε-viniferin, the composition according to the invention may comprise dimers (PAC B1 and B2). Preferentially, the concentration of flavanol dimers (PAC B1 and B2) is greater than 5%, the percentage being given by dry weight relative to the total dry weight of the composition.

When the composition according to the invention comprises at least one grape pomace extract, preferentially the grape pomace extract(s) preferentially comprise flavonols. Preferably, the concentration of flavonols in the composition is greater than or equal to 0.05% by dry weight relative to the total dry weight of the composition.

The composition according to the invention can comprise, in addition to the mixture of molecules described above, at least one other constituent selected from antioxidants, natural extracts, vitamins, trace elements, carotenoids, omega-3 fatty acids, natural oils, and mixtures thereof.

When the composition according to the invention further comprises a natural extract other than the Vitis vinifera extract, the natural extract is preferentially a licorice extract.

Licorice is used in particular in traditional medicine. The first evidence of its documented use in medicine goes back as far as ancient Assyrian, Egyptian, Chinese and Indian cultures.

It is associated with many benefits. Taken orally, licorice was therefore one of the remedies for disorders of the respiratory, gastrointestinal, cardiovascular and urogenital systems. Topically, it was used for the treatment of certain diseases of the eyes or skin (Fiore et al. J Ethnopharmacol 2005 Jul. 14; 99(3):317-24). External, in particular topical, use is also documented. Thus, licorice extract is often incorporated into cosmetic formulations aimed at improving the appearance of the skin.

Preferentially, the licorice extract comprises glycyrrhizic acid and/or glabridin.

By way of example of additional plant extracts, the composition preferentially also comprises at least one extract selected from extracts of coffee, olive leaf, blackcurrant, Polygonum, citrus, French marigold, and mixtures thereof.

The polyphenol(s) optionally present in the composition according to the invention may for example be selected from polyphenols, in particular in the form of plant extracts (extracts from olive leaves or olives, blackcurrant, coffee, Polygonum, citrus, capers), or else vitamin C, in particular in the form of plant extracts (extract of acerola, pomegranate, citrus), vitamin E, vitamin A, in particular in the form of plant extracts, or derivatives thereof, zinc, selenium, and mixtures thereof.

The vitamin(s) optionally present in the composition according to the invention may for example be selected from vitamin B9, B12, C, D, E, A, including pro-vitamins A, derivatives thereof and mixtures thereof.

The trace element(s) optionally present in the composition according to the invention may for example be selected from zinc, selenium or chromium, and mixtures thereof.

Zinc is a mineral essential to good brain functioning by improving the sensitivity thereof to insulin, by reducing oxidative stress and inflammation. Moreover, the European Food Safety Authority, in the context of Regulation 1924/2006, recognizes the role of zinc in normal cognitive function.

The carotenoids(s) optionally present in the composition according to the invention may for example be selected from lutein, zeaxanthin, crocin, picrocrocin and mixtures thereof.

The omega-3 fatty acid(s) optionally present in the composition according to the invention may for example be selected from docosahexaenoic acid (DHA) and eicosapentaenoic acid (EPA) and mixtures thereof.

Long-chain omega-3 fatty acids such as docosahexaenoic acid (DHA) have a beneficial effect on cognitive functions at the preclinical level in aged mice. Its beneficial effects on neuroinflammation are well accepted at the present time and are widely described in the literature.

The natural oil(s) optionally present in the composition according to the invention may for example be selected from fish oils, vegetable oils, microalgae-derived oils and mixtures thereof.

The composition may also comprise other constituents, such as in particular excipients, coating agents such as maltodextrin, microcrystalline cellulose, cyclodextrins, starch, soluble or insoluble fibers, water or alcohol.

According to another subject of the invention, the composition and/or one or more molecules of the composition may be encapsulated or microencapsulated in at least one food support selected from a maltodextrin, a gum arabic, a hydrogenated oil, a non-hydrogenated oil, a wax, an alginate, starch, a protein and mixtures thereof.

The encapsulation is particularly advantageous and makes it possible to stabilize and protect the mixture of molecules according to the invention from its environment, in particular the body of the human or animal that ingested the composition. Thus, the flavanol monomers and ε-viniferin are protected, and their bioavailability is improved.

Furthermore, the encapsulation makes it possible to trap the volatile and heat-sensitive active metabolites in the matrix, to improve the stability, bioavailability and use of said mixture of molecules in food matrices, in particular with masking of the taste and resistance to possible deterioration of the compounds during the steps for producing a food product.

According to one variant, the composition and/or one or more molecules may be microencapsulated. Microencapsulation makes it possible to protect the active substances within particles having sizes of between 1 μm and 1000 μm, more preferentially between 30 μm and 500 μm and even more preferentially between 50 μm and 300 μm.

According to another subject, the composition according to the invention is in the form of a powder, gelcap, tablet, capsule, solution, suspension, emulsion or chewing gum.

When the composition is intended to be used as a nutritional product, food product or dietary supplement, the composition may be in the form of a dairy product, cereals, cereal product or beverage.

Preferentially, the composition according to the invention is delivered at a dose making it possible to provide a daily dose to humans or animals of at least 0.25 mg per kg of body weight of flavanol monomers and 0.025 μg per kg of body weight of ε-viniferin.

### Method

The composition according to the invention can be obtained by any method making it possible to obtain a mixture of molecules comprising at least 15% of flavanol monomers by weight relative to the total weight of the composition and at least 15 ppm of ε-viniferin by weight relative to the total weight of the composition. This may be the mixture of the different molecules in the desired proportions or else the mixture of plant extracts comprising the different molecules in the desired proportions.

According to one embodiment, the composition according to the invention is obtained from a Vitis vinifera extract or from a mixture of Vitis vinifera extracts. The extracts can be obtained by any method making it possible to obtain a mixture comprising at least 15% of flavanol monomers by weight and at least 15 ppm of ε-viniferin by weight.

A particularly suitable method for obtaining a Vitis vinifera extract comprises the following steps:
extraction with water and/or alcohol (in particular ethanol) from Vitis vinifera (e.g. fruit, skins, seeds, leaves, stalks, vine shoots, vine stocks, wood, roots), preferentially from Vitis vinifera seeds and/or Vitis vinifera pomace (the pomace containing grape skins, that is to say the skin of the grapes, grape seeds and optionally stalks), it being possible for the seeds and/or the pomace to originate in particular from the pressing residues from wine production, and/or Vitis vinifera leaves and/or Vitis vinifera vine shoots, vine stocks, wood, roots.

The amount of water or of aqueous-alcoholic solution (30% v/v to 96% v/v) implemented is preferentially between 2 and 10 times the mass of material implemented. The duration of the extraction can be between 30 minutes and 24 hours, and the extraction temperature can be between 20° C. and 80° C.

The raw materials used may be in dry, fresh, or frozen, whole or ground form;
separation of the solution of water and/or alcohol from the solid material, for example by centrifugal separation or by pressing and filtration; the extract obtained in this way is referred to as crude extract A,
it is possible to eliminate the alcohol by vacuum evaporation at a temperature, preferentially of less than 60° C. and at a pressure of less than 100 mbar.

If a plurality of starting materials, for example grape seeds and grape pomace, are used, the method can comprise a single extraction from the two mixed starting materials or else the method can consist in obtaining two separate extracts (implemented separately for each extract over all or part of the method, then mixing, at the end, of the two extracts obtained).

Based on the crude extract(s) A previously obtained, the method may comprise a specific step to make it possible to obtain one or more purified extract(s) rich in flavanol monomers and/or ε-viniferin.

For the flavanol monomers, purification may be carried out either:
by purification by ultrafiltration and/or nanofiltration membrane separation, known to those skilled in the art, of the previously desolvated extract, so as to preferentially select the flavanol monomers and to eliminate the flavanol polymers, in order to obtain an extract comprising a fraction enriched in flavanol monomers,
by purification using liquid/liquid separation using organic solvents, known to those skilled in the art, and in particular using ethyl acetate,
by purification via precipitation, using salts such as NaCl, of the flavanol polymers, followed by solid/liquid separation such as filtration, for example.

The extract thus obtained is a purified extract rich in flavanol monomers, referred to as extract B.

The method preferentially comprises an additional step of chromatographic purification of the extract A and/or of the extract B. This step consists in passing the extracts A and/or B over a chromatographic column containing adsorption resins that are particularly suitable for polyphenols and are known to those skilled in the art, and then rinsing the resins by means of an aqueous solution followed by an elution phase using an aqueous ethanol solution, preferably between 60 and 80% v/v.

The alcohol of the eluates thus obtained is evaporated off and the eluate is concentrated via a step of concentration under vacuum of the eluates that are rich in polyphenols and more particularly in flavanol monomers and/or in ε-viniferin, preferentially at a temperature of less than 60° C. and a pressure of less than 100 mbar.

The method may comprise an additional step which consists in drying the purified extracts using techniques known to those skilled in the art such as spray drying, drying in an oven under vacuum or drying by lyophilization with or without a support such as maltodextrin, gum arabic for example.

The method may also comprise a step of encapsulating or microencapsulating the molecules and/or the composition.

Once the extract has been obtained, the method for preparing the composition according to the invention may comprise mixing extracts of Vitis vinifera rich in flavanol monomers and also in ε-viniferin.

### Use

According to another aspect, the invention also relates to the composition according to any one of the preceding embodiments for use thereof as a drug for humans or animals.

Preferentially, the composition according to the invention is intended to be used in the prevention and/or treatment of diseases associated with endothelial dysfunction, selected from cardiovascular diseases, vascular diseases, diseases associated with cognitive decline, diseases associated with memory loss, neurodegenerative diseases, digestive diseases, joint diseases, eye diseases, erectile disorders and disorders associated with the menopause.

When the composition is intended to prevent and/or treat diseases associated with cognitive decline, including neurodegenerative diseases or diseases associated with memory loss, said disease is selected from Alzheimer's disease, Parkinson's disease, Huntington's disease, motor neurone diseases, dementia, depression, anxiety, schizophrenia, intellectual disability, and cognitive dysfunction syndrome (CDS).

When the composition is intended to prevent and/or treat cardiovascular diseases, said disease is selected from arterial hypertension, atherosclerosis, myocardial infarction, angina pectoris (angina).

When the composition is intended to prevent and/or treat vascular diseases, said disease is selected from chronic venous insufficiency, venous thrombosis, varicose veins.

When the composition is intended to prevent and/or treat digestive diseases, said disease is selected from irritable bowel syndrome, Crohn's disease, ulcerative colitis and dyspepsia.

When the composition is intended to prevent and/or treat joint diseases, said disease is selected from arthritis, rheumatoid arthritis, rheumatoid polyarthritis, ankylosing spondylitis.

When the composition is intended to prevent and/or treat eye diseases, said disease is selected from age-related macular degeneration (AMD), hypertensive retinopathy, diabetic retinopathy, glaucoma, cataracts or Fuchs' endothelial dystrophy.

When the composition is intended to prevent and/or treat sleep disorders, said disorder is selected from insomnia, parasomnia, apnea and/or hypopnea.

When the composition is intended to prevent and/or treat disorders associated with the menopause, said disorder is selected from hot flashes, night sweats, increased abdominal fat, vaginal dryness, drop in libido, mood disorders.

According to another aspect, the invention also relates to a non-therapeutic use of the composition according to the invention in healthy humans or animals, for improving cognitive functions and/or executive functions, and/or for limiting normal age-related cognitive decline and/or for improving memory and/or attention and/or concentration and/or alertness and/or vigilance and/or learning and/or language and/or mood and/or stress and/or anxiety and/or sleep and/or for homogenizing skin pigmentation, and/or for improving the skin's attractiveness and/or for reducing the appearance of age-related skin blemishes, and/or for regulating melanogenesis and/or hyperpigmentation due to excess melanin production, and/or for preventing skin damage and/or disorders associated with sun exposure, such as sunburn and skin aging.

When the composition according to the invention is intended to be used for homogenizing skin pigmentation, and/or improving the skin's attractiveness and/or for reducing the appearance of age-related skin blemishes, and/or for regulating melanogenesis and/or hyperpigmentation due to excess melanin production, and/or for preventing skin damage and/or disorders associated with sun exposure, such as sunburn and skin aging, the composition preferentially comprises at least one licorice extract.

For the purposes of the invention, hyperpigmentation due to excess melanin production means post-inflammatory hyperpigmentation, or hyperpigmentation due to phytophotodermatitis, to a neoplastic process (lentiligos, melanoma), to melasma, to freckles, to acanthosis nigricans, resulting from taking drugs or sun exposure.

The invention will now be illustrated using non-limiting examples of compositions according to the invention, of extract according to the invention, of extract not in accordance with the invention, and using efficacy results.

### Examples

### Example 1: Grape Seed Extract Rich in Flavanol Monomers

The extract is obtained according to the following method:
kg of dry raisin seeds, aqueous extraction in 400 kg of water heated to 85° C.
Liquid/solid separation, then the aqueous extract is passed over a <15 kD membrane
The permeate is then passed over a C18 adsorption chromatography column
Purification over adsorption resin, 10 BV at 2 BV/h,
Washing with water, 4 BV at 2 BV/h
Aqueous ethanol elution with 80% V/V solution at 1 Bv/h with 2 BV
Concentration under vacuum and dealcoholization under 60 mbar at 40° C.
Drying by spray-drying.

The grape seed extract thus obtained comprises at least 15% of flavanol monomers and comprising at least 50% of total polyphenols, measured by the Folin method. Such an extract (E1) is characterized in Table 2 below.

### Example 2: Red Grape Skin Extract Rich in Anthocyanins but without Stilbenes

The extract is obtained according to the following method:
40 kg of red grape pomace
Extraction with 200 kg of aqueous solution containing 0.1% of sulfites
Concentration under vacuum at 100 mbar at 50° C., partial elimination of sulfites
Drying by spray-drying.

The grape extract thus obtained (E2) is characterized in Table 2 below.

### Example 3: Grape Pomace Extract Comprising Stilbenes

The extract is obtained according to the following method:
Aqueous-alcoholic extraction
Red grape pomace extract (E3)
10 kg of dry pomace, aqueous-alcoholic extraction at 30% V/V, solid/liquid ratio 1/10, stirring at 350 rpm at 80° C. for 4 hours
Solid-liquid separation by filtration at 20 μm
Concentration under vacuum at 40° C.
Drying by spray-drying.

The grape extract thus obtained (E3) is characterized in Table 2 below.

### Example 4: Purified Red Grape Pomace Extract Comprising Stilbenes

The extract is obtained according to the following method:
Extraction at 80° C. for 4 h at 50% v/v EtOh, 1/10 ratio
10 kg of dry pomace, aqueous-alcoholic extraction at 30% V/V, solid/liquid ratio 1/10, stirring at 350 rpm at 80° C. for 4 hours
Solid-liquid separation by filtration at 20 μm
Concentration under vacuum at 40° C.
Purification over adsorption resin, 20 BV at 2 BV/h
Washing with water, 4 BV at 2 BV/h
Aqueous ethanol elution with 80% V/V solution at 1 BV/h with 2 BV
Concentration under vacuum and dealcoholization under 200 mbar at 40° C.
Drying by lyophilization

The grape extract thus obtained (E4) is characterized in Table 2 below.

### Example 5: White Grape Pomace Extract Comprising Stilbenes

The extract is obtained according to the following method:
125 kg of white grape pomace, aqueous-alcoholic extraction at 30% V/V, solid/liquid ratio 1/10, stirring at 350 rpm at 80° C. for 4 hours
Solid-liquid separation by filtration at 20 μm
Concentration/dealcoholization under vacuum, 100 mbar at 40° C.
Purification over adsorption resin, 20 BV at 2 BV/h
Washing with water, 4 BV at 2 BV/h
Aqueous ethanol elution with 80% V/V solution at 1 BV/h with 2 BV
Concentration under vacuum and dealcoholization under 200 mbar at 40° C.
Drying by spray-drying.

The grape extract thus obtained (E5) is characterized in Table 2 below.

### Example 6: Vine Shoot Extract Comprising Stilbenes but without Flavanol Monomers

The extract is obtained according to the following method:
40 g of vine shoots, aqueous-alcoholic extraction in 250 g of a 90% V/V solution at 80° C. for 4 h
Solid-liquid separation, filtration
Dealcoholization and concentration under vacuum
Drying by lyophilization.

The Vitis vinifera extract obtained (E6) comprises 5.1% of resveratrol, 8.5% of ε-viniferin, 15.5% of stilbenes and 0% of catechin and epicatechin.

TABLE 2

| | | Red grape | | | | |
|---|---|---|---|---|---|---|
| | | pomace | | | White | |
| | Grape | extract rich | | Purified red | grape | |
| Polyphenols | seed | in | Red grape | grape | pomace | |
| by UHPLC- | extract | anthocyanins | pomace | pomace | extract | Vine shoots |
| DAD-FLD | (E1) | (E2) | extract (E3) | extract (E4) | (E5) | extract (E6) |
| Gallic acid | 0.6634% | 0.1109% | 0.1912% | 0.0609% | 0.0294% | n.a. |
| | | | | | | (<0.0005%) |
| Flavanols | | | | | | |
| Catechin | 21.75% | | 0.47% | 5.69% | 1.22% | n.a. |
| | | | | | | (<0.0005%) |
| Epicatechin | 13.84% | | 0.30% | 3.88% | 0.88% | n.a. |
| | | | | | | (<0.0005%) |
| Total | 35.59% | | 0.77% | 9.57% | 2.10% | n.a. |
| monomers | | | | | | (<0.0005%) |
| other than | | | | | | |
| epicatechin | | | | | | |
| gallate | | | | | | |
| Epicatechin | 2.55% | | 0.17% | 0.7% | 0.53% | n.a. |
| gallate | | | | | | (<0.0005%) |
| Total | 38.14% | | 0.94% | 10.27% | 2.63% | n.a. |
| monomers | | | | | | (<0.0005%) |
| PAC B2 | 4.29% | | 0.19% | 0.89% | 0.28% | n.a. |
| (dimer)* | | | | | | (<0.0005%) |
| PAC B1 | 5.06% | | 0.18% | 1.25% | 0.25% | n.a. |
| (dimer)* | | | | | | (<0.0005%) |
| PAC B2-gallate | 3.78% | | 0.09% | 0.43% | 0.45% | n.a. |
| | | | | | | (<0.0005%) |
| Total PAC | 13.13% | | 0.46% | 2.57% | 0.98% | n.a. |
| dimer | | | | | | (<0.0005%) |
| Flavonols total | n.a. | 0.5249% | 0.4343% | 0.6582% | 0.5829% | n.a. |
| quercetin | (<0.0005%) | | | | | (<0.0005%) |
| derivatives | | | | | | |
| Stilbenes | | | | | | |
| trans- | n.a. | n.a. | 0.0146% | 0.1035% | 0.1146% | 5.34% |
| Resveratrol | (<0.0005%) | (<0.0005%) | | | | |
| Piceid | n.a. | n.a. | n.a. | n.a. | 0.0343% | 0.0513% |
| | (<0.0005%) | (<0.0005%) | (<0.0005%) | (<0.0005%) | | |
| Piceatannol | n.a. | n.a. | n.a. | n.a. | 0.0589% | 0.1424% |
| | (<0.0005%) | (<0.0005%) | (<0.0005%) | (<0.0005%) | | |
| e-Viniferin | n.a. (<5 ppm) | n.a. (<5 ppm) | 61 ppm | 529 ppm | 5962 ppm | 85600 ppm |
| R-Viniferin | n.a. | n.a. | 0.0035% | 0.0159% | 0.0392% | 1.4383% |
| | (<0.0005%) | (<0.0005%) | | | | |
| Viniferin 2 | n.a. | n.a. | n.a. | 0.0041% | 0.1130% | 1.2818% |
| | (<0.0005%) | (<0.0005%) | (<0.0005%) | | | |
| Viniferin 3 | n.a. | n.a. | 0.0200% | n.a. | 0.0517% | 0.4347% |
| | (<0.0005%) | (<0.0005%) | | (<0.0005%) | | |
| Viniferin 5 | n.a. | n.a. | 0.0135% | n.a. | 0.1110% | 0.4712% |
| | (<0.0005%) | (<0.0005%) | | (<0.0005%) | | |
| Total | n.a. | n.a. | 0.0577% | 0.1734% | 1.1189% | 17.7572% |
| Stilbenoids | (<0.0005%) | (<0.0005%) | | | | |
| Total | 99.6% | 31.1% | 36.8% | 57.9% | 55.3% | 53.3% |
| Polyphenols | | | | | | |
| (Folin) | | | | | | |
| Ratio of | 35.73% | — | 2.1% | 16.5% | 3.8% | 0 |
| flavanol | | | | | | |
| monomers to | | | | | | |
| total | | | | | | |
| polyphenols | | | | | | |

### Example 7: Examples of Compositions According to the Invention

Table 3 below presents several examples of compositions comprising extracts originating from the methods described above in examples 1 to 6.

TABLE 3

| Examples | | | Flavanol | | | | | |
|---|---|---|---|---|---|---|---|---|
| according | | | dimers | | | Ratio of | | P |
| to the | | Flavanol | (PAC B2 | | | monomers/ | | Total |
| invention | Formulation | monomers | and B1) | ε-viniferin | Stilbenes | ε-viniferin | Flavonols | polyphenols |
| A | 75% E1 + 25% E3 | 26.9% | 10.0% | 0.0015% | 0.0144% | 17639/1 | 0.11% | 83% |
| B | 75% E1 + 25% E4 | 29.1% | 10.6% | 0.0132% | 0.0434% | 2200/1 | 0.16% | 74% |
| C | 99% E1 + 1% E5 | 35.3% | 9.8% | 0.0060% | 0.0112% | 6272/1 | 0.05% | 99% |
| D | 40% E1 + 60% E5 | 15.5% | 5.3% | 0.3577% | 0.6713% | 43/1 | 0.34% | 82% |
| E | 40% E1 + 35% | 15.5% | 5.4% | 0.0015% | 0.0144% | 10160/1 | 0.11% | 60% |
| | E2 + 25% E3 | | | | | | | |
| F | 50% E1 + 50% E6 | 17.8% | 6.6% | 4.2800% | 8.8700% | 4.15/1 | — | 76% |

It should be noted that the concentration of flavanol dimers (PAC B1 and B2) is greater than 5% by dry weight of the weight of the composition. In addition, for the Vitis vinifera extracts derived from the parts of the pomace (seed, skin, stalks), the concentration of flavonols is equal to or greater than 0.05% by dry weight of the weight of the composition.

### Example 8: Evaluation of the Effect of the Composition According to the Invention on Tyrosinase Activity

### Evaluation of the Effect of a Composition Comprising Flavanol Monomers and ε-Viniferin

The aim of this test is to model the activity of an enzyme, tyrosinase (the enzyme responsible for melanin formation) using an experimental design and to determine the influence of flavanol monomers and ε-viniferin on enzymatic activity with respect to a specific substrate (L-DOPA). Tyrosinase activity was measured in vitro using a colorimetric reaction. Under the action of tyrosinase, L-DOPA is oxidized and converted into dopaquinone. The formation of dopaquinone can thus be monitored by spectrometry at 475 nm.

In a 96-well plate, the following amounts were deposited in each well: 80 μl phosphate buffer (50 mM), 40 μl of sample to be tested or buffer, 40 μl of tyrosinase at 125 U/ml. At the last moment, 40 μl of L-DOPA (2.5 mM) was added to initiate the reaction and then absorbance at 475 nm was monitored for 25 min.

For each well, the absorbance values over time (Aₓ) are normalized relative to the first absorbance measurement (A_{T0}) according to the following formula: Normalized absorbance (Aₙ)=Aₓ−A_{T0}. The normalized absorbances are then corrected (for each time) according to the following formula: Corrected normalized absorbance (A_{nc})=Aₙ−A_{CLT}, where A_{CLT} is the value of the normalized absorbance of the reagents in the presence of the sample (buffer+tyrosinase+sample). The lower the observed values of (A_{nc}), the greater the reduction in tyrosinase activity (with respect to the L-DOPA substrate).

To perform this test, design expert software was used, and a central composite design was used as an experimental design, using the factors of: concentrations of between 1 and 50 μM for the flavanol monomers and between 0.0050 and 0.05 μM for the ε-viniferin, and, for the response, the corrected normalized absorbance measured after 7 min. The ratio of flavanol monomers to ε-viniferin is between 10,000 and 1,000.

The results obtained showed that the corrected normalized absorbance could be modeled using a linear-quadratic model (p<0.0001) (Table 4).

TABLE 4

| | Sum of | | Root mean | | |
|---|---|---|---|---|---|
| Source | squares | df | square | F-value | p-value |
| Model | 0.0349 | 5 | 0.0070 | 27.53 | <0.0001 |
| A-Flavanols | 0.0275 | 1 | 0.0275 | 108.46 | <0.0001 |
| B-ε-Viniferin | 0.0005 | 1 | 0.0005 | 1.88 | 0.1780 |
| AB | 0.0023 | 1 | 0.0023 | 9.00 | 0.0047 |
| A² | 0.0030 | 1 | 0.0030 | 11.76 | 0.0015 |
| B² | 0.0006 | 1 | 0.0006 | 2.42 | 0.1285 |
| Residual | 0.0096 | 38 | 0.0003 | | |
| Lack of Fit | 0.0019 | 3 | 0.0006 | 2.86 | 0.0509 |
| | Value | | | | |
| R² | 0.7837 | | | | |
| Adjusted R² | 0.7552 | | | | |
| Predicted R² | 0.7025 | | | | |

The results also demonstrate a significant effect of the flavanol monomers on tyrosinase activity with respect to the L-DOPA substrate. The analysis of the coefficients in terms of coded factors (Table 5) shows that the flavanol monomers reduce tyrosinase activity with respect to its substrate, L-DOPA.

TABLE 5

| | Estimated | | Standard | 95% CI | 95% CI |
|---|---|---|---|---|---|
| Factor | Coefficient | df | error | (lower) | (upper) |
| Intercept | 0.1508 | 1 | 0.0041 | 0.1426 | 0.1591 |
| A-Flavanols | −0.0338 | 1 | 0.0032 | −0.0404 | −0.0273 |
| B-ε-Viniferin | −0.0045 | 1 | 0.0032 | −0.0110 | 0.0021 |
| AB | −0.0119 | 1 | 0.0040 | −0.0200 | −0.0039 |
| A² | 0.0171 | 1 | 0.0050 | 0.0070 | 0.0273 |
| B² | 0.0078 | 1 | 0.0050 | −0.0024 | 0.0179 |

In contrast, no significant effect of the ε-viniferin was observed in the concentration range tested in the experimental design. Finally, interestingly, a significant interaction between the flavanol monomers and the ε-viniferin was observed. In addition, since the coefficient of the interaction of these factors is negative, this demonstrates that the association of flavanol monomers and ε-viniferin also reduces tyrosinase activity with respect to the L-DOPA substrate.

The effect of the combination of flavanol monomers and ε-viniferin is greater than an additive effect of the molecules. Indeed, for a concentration of flavanol monomers of 35 μM and a concentration of ε-viniferin at 0.005 μM, an absorbance value of 0.158 is obtained, while the absorbance value for an additive effect is 0.332. Consequently, the composition according to the invention does indeed exhibit a synergistic effect.

### Evaluation of the Effect of a Mixture of Vitis vinifera Extracts and Licorice

### Extract

The aim of this test is to evaluate tyrosinase activity (with respect to the L-DOPA substrate) in the presence of a mixture comprising:
58.8% of a composition according to the invention, itself composed of 53.4% of E1 and 46.6% of E3 (the composition according to the invention providing 19.4% of flavanol monomers and 28 ppm of ε-viniferin) and
41.2% of a licorice extract providing 8.1% of glycyrrhizic acid.

This activity will be compared to enzymatic activities in the presence of E1 or E3 or licorice extract alone.

To this end, tyrosinase activity was monitored and measured in vitro using the colorimetric reaction described above. The extracts were tested at the following concentrations: grape seed extract at 0.028 mg/ml, red grape pomace extract at 0.025 mg/ml, licorice extract at 0.037 mg/ml.

The percentage tyrosinase inhibition was determined on the normalized and corrected absorbances, at T=7 min, according to the following equation.

$$\% inhibition = \frac{{A}_{nc} control - {A}_{nc} sample}{{A}_{nc} control} \times 100$$

Where A_{nc} Control is the mean of the normalized and corrected absorbance measurements of the standard reactions (buffer+tyrosinase+L-DOPA) and A_{nc} Sample is the corrected normalized absorbance of the reaction in the presence of the sample (buffer+tyrosinase+L-DOPA+sample).

The absorbance values (at T=7 min) were compared statistically using a 3-way analysis of variance (ANOVA), where factor 1 was treatment with a grape seed extract, factor 2 was treatment with a red grape pomace extract, factor 3 was treatment with a licorice extract. A value of p<0.05 is considered significant.

In the presence of a red grape pomace extract, a low and non-significant reduction in tyrosinase activity was observed (FIG. 1). In the presence of a licorice extract or a grape seed extract, significant reductions of 40.2% and 49.8%, respectively, in tyrosinase activity were observed. Finally, a significant interaction was found between a seed extract, a red grape pomace extract and a licorice extract. Thus, such a mixture according to the invention exhibited a greater reduction (84.9%) in tyrosinase activity than that observed for red grape pomace extracts, a grape seed extract or a licorice extract alone. Reducing tyrosinase activity with respect to the L-DOPA substrate could thus make it possible to reduce melanin production and consequently improve skin homogeneity and reduce age-related pigmentation blemishes or hyperpigmentation processes.

### Example 9: Evaluation of the Effect of a Composition According to the Invention on Endothelial Function

This test aims to evaluate the vasorelaxation, ex vivo, of rat aortas, in the presence of a mixture comprising:
65% of a composition according to the invention, itself composed of 92.3% of E1 and 7.7% of E5 (the composition according to the invention providing 33% of flavanol monomers and 459 ppm of ε-viniferin, and
35% of maltodextrin.

The vasorelaxation induced by this mixture will be compared with the vasorelaxation induced by extracts E1 and E5 alone.

To this end, rat thoracic aortas were sampled, cleaned and sectioned into 2-3 mm rings. The rings were then submerged in an isolated organ bath, in 20 ml of Krebs solution (118 mM NaCl, 25 mM NaHCO₃, 5.5 mM d-glucose, 4.7 mM KCl, 1.18 mM KH2PO4, 2.4 mM MgSO4, and 3.3 mM CaCl2), pH 7.8) at 40° C. and continuously bubbled with 95% 02 and 5% CO2. To test the reactivity of the endothelium, contractions were induced with KCl (80 mM) until a plateau was reached. The organ bath was then washed 3 times with Krebs solution. The functional endothelium was then tested by first adding 0.5% noradrenaline (at 0.1 μM), to induce contraction, then acetylcholine (at 10 μM), to induce at least 80% vasorelaxation. Cumulative sample volumes were then added to the organ bath to reach a final volume of 2000 μl over a period of 1 h and a concentration-relaxation curve was constructed. Three independent tests were performed for each sample.

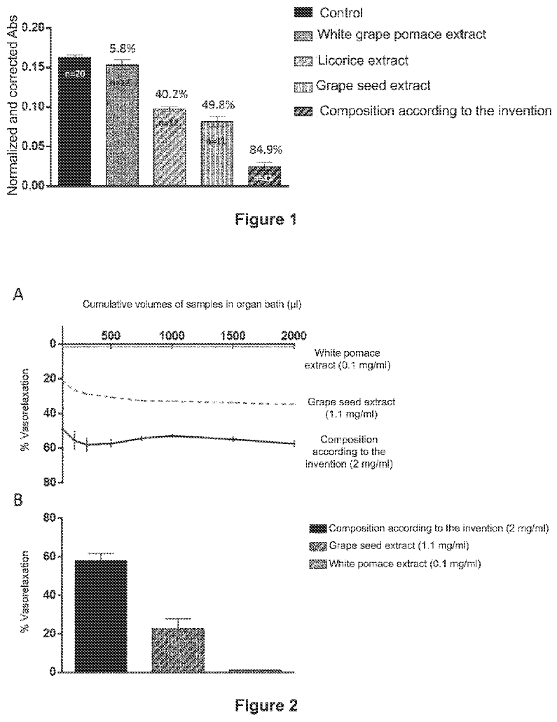

The results show that the mixture comprising a composition according to the invention induces strong vasorelaxation of the aortic sections (FIG. 2A). Indeed, the maximum observed vasorelaxation is 58% and is reached after adding 300 μl of sample. In the presence of the mixture comprising a composition according to the invention, the effect on vasorelaxation is greater than that observed for a grape seed extract alone (and at the same concentration as in the mixture comprising a composition according to the invention) and than that estimated for a white grape pomace extract alone (and at the same concentration as in the mixture comprising a composition according to the invention) (FIG. 2B). The mixture comprising a composition according to the invention also induces a greater effect than the cumulative effects of the extracts (not in accordance with the invention) taken separately. These results demonstrate that there are synergistic interactions between the flavanol monomers and ε-viniferin in the mixture comprising a composition according to the invention. Such a synergistic effect of the composition of the invention makes it possible to propose formulations that improve endothelial function and therefore vascular function.

### Example 10: Evaluation of the Antioxidant Capacity of a Mixture of Vitis vinifera Extracts

The aim of this test is to determine the antioxidant activity of Vitis vinifera extracts such as grape seed extract or a mixture of Vitis vinifera extracts comprising 50% of E1 and 50% of E3, by comparing it with a vitamin E analog, Trolox. Antioxidant activity was measured, by spectrophotometry, using the oxygen radical absorbance capacity (ORAC) assay. This assay is based on the fact that the fluorescence of the molecule fluorescein decreases under the action of an oxidizing agent, AAPH (2,2′-azobis(2-amidinopropane) dihydrochloride), which generates peroxide radicals. The presence of antioxidants prevents or slows the measured fluorescence loss which is calculated by the area under the curve as a function of time. The antioxidant capacity of the sample tested is then expressed as Trolox equivalents, that is to say as a function of the concentration of Trolox having the same activity as the tested sample at a given concentration. The ORAC value obtained is independent of the concentration tested for the sample.

In a 96-well plate, the following amounts were deposited in each well: 25 μl of phosphate buffer (50 mM, pH 7.5) for the blanks or 25 μl of Trolox (25 μM, 20 μM, 15 μM, 10 μM, 5 μM, 2 μM), 25 μl of diluted sample, 150 μl of fluorescein (8 nM). The plate was then incubated for 30 min at 37° C. Just before taking the reading, 25 μl of AAPH (153 mM) were added.

The results show that grape seed extracts and also the mixture of Vitis vinifera extracts have a strong antioxidant capacity, with ORAC values of between 10524 and 24142. Thus, a mixture of Vitis vinifera extracts according to the invention makes it possible to propose formulations with a strong antioxidant power. Given that it is recommended to consume 3000 to 5000 ORAC units every day in order to reduce oxidative stress, such a composition according to the invention would make this possible.

### Example 11: Evaluation of the Antioxidant Effect, when Exposed to UVB Radiation, of a Mixture of Vitis vinifera Extracts and a Licorice Extract

The aim of this test is to compare the production of reactive oxygen species (ROS) by keratinocytes following chronic exposure to UVB radiation in the presence of a composition according to the invention with that of a control.

To this end, fragments of fresh human skin were immediately cut into 5×5 mm pieces and treated with trypsin-EDTA for 3 h at 37° C., or overnight at 4° C., to separate the epidermis from the dermis. The keratinocytes were seeded at a concentration of 1×10⁵ cells per cm² in 75 cm² flasks in KGM-2 medium, which comprises hydrocortisone (0.33 μg/ml), epidermal growth factor EGF (0.125 ng/ml), insulin (5 μg/ml), bovine pituitary extract (4 μl/ml), epinephrine (0.39 μg/ml) and transferrin (10 μg/ml). When the cultures reached 70-80% confluence, the cells were detached with 10% trypsin-EDTA, then resuspended in supplemented KGM-2 medium and counted on a Malassez counting chamber. The keratinocytes were then cultured at an amount of 5×10⁴ cells per well in a 12-well plate for the test for measuring cytoplasmic ROS production via a chloromethyl H2DCFDA derivative, CM-H2DCFDA. Three days before UVB irradiation, a mixture comprising a composition according to the invention was placed in the presence or absence of the cells. The mixture tested was as follows:
53.7% of a composition according to the invention, itself composed of 53.4% of E1 and 46.6% of E3 (the composition according to the invention providing 19.4% of flavanol monomers and 28 ppm of ε-viniferin) and
37.5% of a licorice extract.

After changing the culture medium, the cells were or were not irradiated a 1st time at 60 mJ/cm², then new medium, in the presence or absence of the mixture of extracts, was added and the cells were returned to the oven at 37° C. Twenty-four hours after the 1st irradiation, the culture medium was changed and the cells were or were not irradiated a 2nd time at 60 mJ/cm², then new medium, in the presence or absence of the mixture of extracts, was added and the cells were returned to the oven at 37° C. Twenty-four hours after the 2nd irradiation, the culture medium was changed and the cells were or were not irradiated a 3rd time at 60 mJ/cm², then new medium, in the presence or absence of the mixture of extracts, was added and the cells were returned to the oven at 37° C. for 30 min.

Thirty minutes after the 3rd and final irradiation, the cells were detached from the culture wells by treatment with trypsin-EDTA, washed then incubated with 1 μM/tube of CM-H2DCFDA for 30 minutes, at 37° C. and in the dark. After this incubation, the cells were washed twice in PBS and then analyzed by flow cytometry. A minimum of 1×10⁴ cells were collected and analyzed by testing. CM-H2DCFDA makes it possible to indicate reactive oxygen species (ROS) derivatives in the cells. It diffuses passively into cells, where its acetate groups are cleaved by intracellular esterases and its thiol-reactive chloromethyl group reacts with intracellular glutathione and with other thiols. The resulting oxidation produces a fluorescent adduct, which is trapped inside the cell.

The mean fluorescence intensity values per cell were compared statistically using a two-way analysis of variance (ANOVA), where factor 1 was treatment with the mixture of extracts or not, factor 2 was irradiation treatment or not. A value of p<0.05 is considered significant.

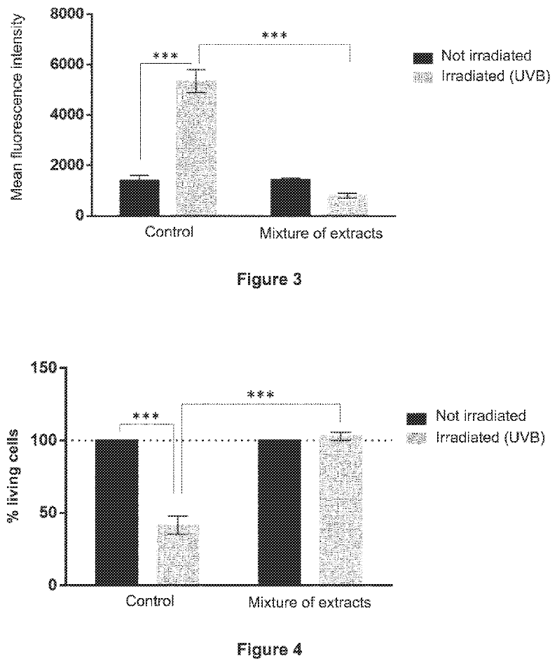

The results show that chronic irradiation at 60 mJ/cm² induces oxidative stress and significant ROS production. Indeed, the mean fluorescence intensity per cell significantly increases from 1389 to 5346 after exposure to UVB. In contrast, in the presence of a mixture of Vitis vinifera extract and a licorice extract, no increase in the mean fluorescence intensity was observed, which means that there was no cytoplasmic production of ROS (FIG. 3).

These results demonstrate the antioxidant power and the protective effect of a mixture of Vitis vinifera extract and a licorice extract when exposed to UVB irradiation.

### Example 12: Evaluation of Cell Viability Following UVB Exposure and in the Presence of a Mixture of Vitis vinifera Extracts and a Licorice Extract

The aim of this test is to compare the keratinocyte proliferation rate following chronic exposure to UVB radiation in the presence of a composition according to the invention with that of a control.

After isolating the keratinocytes, they are cultured at an amount of 5×10³ cells per well in 96-well plates. Three days before UVB irradiation, a mixture comprising a composition according to the invention was placed in the presence or absence of the cells. The mixture tested was as follows:
53.7% of a composition according to the invention, itself composed of 53.4% of E1 and 46.6% of E3 (the composition according to the invention providing 19.4% of flavanol monomers and 28 ppm of ε-viniferin) and
37.5% of a licorice extract.

After changing the culture medium, the cells were or were not irradiated a 1st time at 60 mJ/cm², then new medium, in the presence or absence of the extracts, was added and the cells were returned to the oven at 37° C. Twenty-four hours after the 1st irradiation, the culture medium was changed and the cells were or were not irradiated a 2nd time at 60 mJ/cm2, then new medium, in the presence or absence of the extracts, was added and the cells were returned to the oven at 37° C. Twenty-four hours after the 2nd irradiation, the culture medium was changed and the cells were or were not irradiated a 3rd time at 60 mJ/cm², then new medium, in the presence or absence of the extracts, was added and the cells were returned to the oven at 37° C.

Twenty-four hours after the 3rd and final irradiation, 20 μl of MTS were added to the wells, then the cells were returned to the oven at 37° C. and under 5% CO2 for 3 h. At the end of of the 3 h, the absorbance (OD) was read at 490 nm by a spectrophotometer. The OD values are then expressed as a percentage relative to the non-irradiated control for each condition.

The percentages of cell viability were compared statistically using a two-way analysis of variance (ANOVA), where factor 1 was treatment with the mixture of extracts or not, factor 2 was irradiation treatment or not. A value of p<0.05 is considered significant.

The results show that chronic irradiation at 60 mJ/cm² induces a significant reduction in the number of viable cells. Indeed, the survival rate after exposure to UVB is 41.5%.

In contrast, in the presence of a mixture of Vitis vinifera extract and a licorice extract, no significant reduction in the number of living cells was observed after UVB exposure (FIG. 4).

These results demonstrate the protective effect of a composition according to the invention on cell viability following UVB irradiation.

### Example 13: Examples of Compositions Intended for Humans in Different Forms

Examples of compositions according to the invention are presented in Table 6 below.

TABLE 6

| | | | | | Meno- |
|---|---|---|---|---|---|
| | Skin application | | | Sight | pause |
| | Exam- | Exam- | Exam- | applica- | Applica- |
| Ingredients | ple 1 | ple 2 | ple 3 | tion | tion |
| Grape seed | 140 mg | 140 mg | 140 mg | 95 mg | 115 mg |
| extract | | | | | |
| Red grape | 50 mg | 50 mg | 50 mg | 35 mg | 40 mg |
| pomace | | | | | |
| extract | | | | | |
| Licorice | 93.5 mg | 93.5 mg | 93.5 mg | | |
| extract | | | | | |
| Vitamin C | 16.5 mg | 16.5 mg | 16.5 mg | | |
| Glutathione | | 50 mg | 50 mg | | |
| Superoxide | | | 3.5 mg | | |
| Dismutase | | | | | |
| Zinc | | | 16.5 mg | 5 mg | |
| Saffron | | | | 20 mg | 30 mg |
| extract | | | | | |
| Vitamin A | | | | 1 mg | |
| Vitamin B6 | | | | | 1 mg |
| Total | 300 mg | 350 mg | 370 mg | 156 mg | 186 mg |
| Form of | Gelcap | Tablet | Sachet | Gelcap | Chewing |
| administration | | | | | gum |

### Example 14: Nutritional Beverage Intended for Humans

Example 14 is a 50 ml beverage containing the equivalent of 300 mg of a mixture of Vitis vinifera extracts. The mixture may be composed of 225 mg of grape seed extract and 75 mg of white grape pomace extract. The extracts may be microencapsulated. The recommended dosage is 1 to 2 shots per day.

### Example 15: Nutrient-Enriched Oil Intended for Humans

Example 15 is a vegetable oil consisting of grape seed oil containing Vitis vinifera extracts and carotenoids. A daily dose of 20 ml contains 150 mg of a mixture of Vitis vinifera extracts and 20 mg of zeaxanthin.

### Example 16: Hyperprotein Powdered Preparation

Example 16 is a food product formulation intended for special medical purposes or a protein-enriched food formulation. The composition is detailed in Table 7. The dosage may for example be one daily dose of said powdered preparation. Said dose may be diluted in a beverage or a meal and represents approximately 12 g.

### Example 17: Yogurt

Example 17 is a formulation in the form of a yogurt, to be consumed 2 to 3 times a day. The composition is detailed in Table 7.

TABLE 7

| Composition according to the invention | Example 16 | Example 17 |
|---|---|---|
| Portion (g) | 100 | 125 |
| Constituents | | |
| Catechin + epicatechin (mg), | 50 | 62.5 |
| that is to say 175 mg of B | | |
| (Table 3) | | |
| ε-viniferin (mg), that is to say | 0.0231 | 0.0289 |
| 175 mg of B (Table 3) | | |
| Others | | |
| Proteins (g) | 83 | 10 |
| Vitamin D (μg) | 125 | 15 |
| Vitamin E (mg) | 167 | 20 |
| Vitamin B9 (μg) | 3333 | 400 |
| Vitamin B12 (μg) | 833 | 100 |
| Vitamin C (mg) | 500 | 60 |
| Zinc (mg) | 42 | 5 |

### Example 18: Kibble for Dogs

Example 18 is a formulation in the form of kibble intended for a 10 kg dog, fed with 200 g of kibble/day, providing:
0.450 mg of catechin and/or epicatechin per kg of body weight, and
0.045 μg of ε-viniferin per kg of body weight.

1. A composition comprising a mixture of molecules, comprising:
at least 15% of flavanol monomers, the percentage being given by dry weight
relative to the total dry weight of the composition, and
at least 15 ppm of ε-viniferin, by dry weight relative to the total dry weight of the composition,
said mixture of molecules is obtained from at least one extract or a mixture of extracts of Vitis vinifera.

2. The composition according to claim 1, said composition comprising at least 100 ppm of stilbenes comprising said at least 15 ppm of ε-viniferin.

3. The composition according to claim 1, characterized in that the amount by weight of flavanol monomers is greater than the amount by weight of ε-viniferin.

4. The composition according to claim 1, characterized in that the ratio of flavanol monomers to ε-viniferin is between 20,000 and 4.

5. The composition according to claim 1, characterized in that it comprises flavanol dimers (PAC B1 and B2) and in that the concentration of flavanol dimers (PAC B1 and B2) is greater than 5%, the percentage being given by dry weight relative to the total dry weight of the composition.

6. The composition according to claim 1, characterized in that the mixture of molecules is obtained from one or more natural product(s) selected from a Vitis extract, a tea extract, an apple extract, a cocoa extract, an iris, Sophora, Gnetum, Carex, peony, Dipterocarpus extract and a microalgae extract.

7. The composition according to claim 6, characterized in that the Vitis vinifera extract comprises at least 50% total polyphenols, the percentage being given by dry weight relative to the total weight of the Vitis vinifera extract.

8. The composition according to claim 1, characterized in that the mixture of molecules is obtained at least from a grape pomace extract and/or from a grape seed extract.

9. The composition according to claim 8, characterized in that the grape pomace extract(s) comprise flavonols and in that the concentration of flavonols in the composition is greater than or equal to 0.05% by dry weight relative to the total dry weight of the composition.

10. The composition according to claim 1, characterized in that it also comprises a licorice extract.

11. The composition according to claim 1, characterized in that the composition and/or one or more molecules of the composition is (are) encapsulated or microencapsulated in at least one food support selected from a maltodextrin, a gum arabic, a hydrogenated oil, a non-hydrogenated oil, a wax, an alginate, starch, a protein and mixtures thereof.

12. The composition according to claim 1, characterized in that it is in the form of a powder, gelcap, tablet, capsule, solution, suspension, emulsion or chewing gum.

13. The composition according to claim 1, characterized in that the composition is a food product in the form of a dairy product, cereals, cereal product or beverage.

14. The composition according to claim 1, for use thereof as a drug for humans or animals.

15. The composition according to claim 14, for use thereof in the prevention and/or treatment of diseases associated with endothelial dysfunction, selected from cardiovascular diseases, vascular diseases, diseases associated with cognitive decline, diseases associated with memory loss, neurodegenerative diseases, digestive diseases, joint diseases, erectile diseases and disorders associated with the menopause.

16. The composition according to claim 15, for use thereof in the prevention and/or treatment of a disease selected from Alzheimer's disease, Parkinson's disease, Huntington's disease, motor neuron diseases, dementia, depression, anxiety, schizophrenia, intellectual disability, cognitive dysfunction syndrome (CDS), arterial hypertension, atherosclerosis, myocardial infarction, angina pectoris (angina), chronic venous insufficiency, venous thrombosis, varicose veins, irritable bowel syndrome, Crohn's disease, ulcerative colitis and dyspepsia, arthritis, rheumatoid arthritis, rheumatoid polyarthritis, ankylosing spondylitis, age-related macular degeneration (AMD), hypertensive retinopathy, diabetic retinopathy, glaucoma, cataracts or Fuchs' endothelial dystrophy.

17. A non-therapeutic use of a composition according to claim 1 in healthy humans or animals, for improving cognitive functions and/or executive functions, and/or for limiting normal age-related cognitive decline and/or for improving memory and/or attention and/or concentration and/or alertness and/or vigilance and/or learning and/or language and/or mood and/or stress and/or anxiety and/or sleep and/or for homogenizing skin pigmentation, and/or for reducing the appearance of age-related skin blemishes and/or for regulating melanogenesis.
